(19) United States
(12) Patent Application Publication
(10) Pub. No.: US 2024/0390043 A1
(43) Pub. Date: Nov. 28, 2024

(54) SYSTEM FOR THE SURGICAL TREATMENT OF FRACTURES SUCH AS PROXIMAL HUMERUS FRACTURES AND INCLUDING A NOVEL DRILL GUIDE, OPTIONAL INTRAMEDULLARY EXPANDABLE BONE ANCHOR AND REDUCTION DRILL GUIDE

(71) Applicant: Shahryar AHMADI, Huntington Beach, CA (US)

(72) Inventor: Shahryar AHMADI, Huntington Beach, CA (US)

(21) Appl. No.: 18/671,312

(22) Filed: May 22, 2024

Related U.S. Application Data
(60) Provisional application No. 63/468,154, filed on May 22, 2023.

(51) Int. Cl. A61B 17/80; A61B 17/17; A61B 17/72

(52) U.S. Cl. CPC A61B 17/8038; A61B 17/1728; A61B 17/7258; A61B 17/8061

(57) ABSTRACT

The present invention provides a system for fixation of fractures comprising an orthopedic plate configured to abut and support the side of a bone. A cupped drill guide cooperates with the end of the plate and envelops a portion of the bone with a hook and drill guide assembly that can be used to reduce that bone. An expandable cage member can be inserted through the plate into the bone to buttress it, to inhibit subsidence, and to cause or to support the reduction. Optionally the cage member holes can be used for a smaller diameter screw through the use of inserts.

### FIELD OF THE INVENTION

The present invention relates generally to the field of orthopedic implants and surgery that can be used for many different bones, and more particularly to a system of implants designed for use in proximal humerus fractures, and including an orthopedic plate and associated drill guide for reduction of a common fracture to the humeral head, as well as an optional intramedullary expandable bone anchor to provide increased stability where and a method of use of the system which can be used to aid in facture reduction, proper union, and to reduce the incidence of malunion and fracture displacement, including varus displacement.

### BACKGROUND OF THE INVENTION

The proximal humerus includes a head that cooperates with the glenoid cavity to form the highly mobile shoulder joint. The structure and mechanics of the shoulder including the skeletal system and the highly complex system of ligaments, tendons and muscles is both beautiful and intricate in design and function. The clavicle, acromion, and coracoid process act, in conjunction with the humeral shaft and head, and the fossa of the scapula, known as the glenoid cavity to form the skeletal structure, while a fibrocartilage rim, the glenoid labrum, the joint capsule of the shoulder, along with various humeral and associated acromial ligaments act in union with the rotator cuff muscles (i.e., supraspinatus, infraspinatus, subscapularis, and teres minor), the deltoids and trapezius, the pectorals major and minor, the biceps, the triceps, coracobrachialis, to stabilize and motivate the shoulder joint.

However, while the complication of this joint contributes to its wide range of functionality, it also contributes to a vulnerability. This is particularly true in an aging population prone to muscle loss, poor quality bone such as osteopenia and osteoporosis, comorbidities, loss of balance, gait uncertainties, and visual impairment. Thus, proximal humeral fractures are commonly seen as the result of a ground level fall on an out-stretched arm, particularly for women 65 and older, as well as for a younger more gender neutral population as a result of high-energy trauma, such as a vehicle accident or fall from height. This injury is the third most common fracture, and most often involves a two-to-four-part fracture located at one or more of the surgical neck, the anatomic neck, or the greater or lesser tuberosities, with a two part surgical neck fracture being the most common. Unsuccessful or incomplete healing carries the risk of loss of nerve or motor function in the affected arm, which can lead to muscular atrophy, loss of function and a spiraling array of social consequences, particularly for an elderly patient who is already at risk of loss of independence. However, in spite of the prevalence of this injury, and of the quality-of-life issues that threaten patients who suffer from poor recovery to a proximal head fracture, medical science has not been successful to date in providing a suitable solution to these difficult injuries.

Fixation of the proximal humerus fracture has always been a challenge. Often the fractures are left to mend without surgery, with varying degrees of success. These patients typically have minimally displaced surgical and anatomic neck fractures. Depending on a number of variables apart from the indication, including the fracture location and degree of displacement, as well as age, fracture type, bone quality, dominance, general medical condition and concurrent injuries. In these instances, a sling is used for immobilization followed by progressive rehabilitation where more immediate physical therapy generally results in faster recovery.

Fractures of the neck, head or intertrochanter of the femur have been successfully treated with a variety of compression screw assemblies, which includes for example, a compression plate having a barrel member, a lag screw and a compressing screw. The compression plate is secured to the exterior of the femur and the barrel member is inserted into a predrilled hole in the direction of the femoral head. The lag screw which has a threaded end and a smooth portion is inserted through the barrel member so that it extends across the break and into the femoral head. Femoral shaft fractures have also been treated with the help of intramedullary rods or “nails” inserted into the marrow canal of the femur to immobilize the femur parts involved in fractures. While these prior art devices have been used to treat femoral fractures and have included means to induce compression in the associated bone in order to facilitate fusion of the bone segments, the differences between the anatomy of the hip and shoulder are sufficiently different to such that the femoral devices are not readily changed to suit use in the humerus.

A number of different surgical methods have been used to address the problem in the humerus, including CRPP (Closed Reduction Percutaneous Pinning), with outcomes having high complication rates including loss of reduction and the potential for nerve, vein, or tendon damage from associated pins. Another method of stabilization has included ORIF (Open Reduction Internal Fixation) which includes the use of bone screws and pins, orthopedic plates and wire. Other known methods of fixation include the use of intramedullary nails positioned in the intramedullary canal of fractured bone, particularly for younger patients with favorable bone quality, and arthroplasty including hemiarthroplasty particularly for younger patients, and reverse total shoulders for low-demand elderly patients, those with poor bone, or older patients with fracture dislocation.

However, for all of these surgical methods of treatment, surgeons' struggle during the operation to be able to reduce the fracture. Also, failure of the fixation is very common and proximal humerus fracture mal-union or non-union is prevalent, especially in some fracture patterns. Up to this point there have multiple design attempts to prevent the failure of fixation but they have had little success. Often if there is no calcar (the medical neck bone), the humeral head will fall into varus, and while screws have been used to try to prevent this subsidence, it has not been very successful.

The present system involves various inventions which function independently and synergistically in cooperation to aid in the execution of various orthopedic surgeries, and specifically including proximal humeral surgeries. The present invention aids in improved outcomes in these surgeries.

### SUMMARY OF THE INVENTION

The invention relates to a system and method of surgery using the system for treating a fracture, and specifically a proximal humeral fracture among other fractures. The system includes a lateral orthopedic plate which is placed on the proximal end of the humerus, and a drill guide that can be used adjacent the plate, including screwed to it, where the drill guide includes an inwardly curving hook on the far side corresponding to the most medial point of the guide body. Thus, the drill guide body, hook, and associated threaded k-wires secure the tuberosity of a proximal humerus. The system further includes means to fix the plate to the humerus, such as screws, pins, bolts and k-wires used during the procedure. These means may include regular screws, locking, or variable locking screws.

In addition, the drill guide includes a drill guide body that has one or more threaded through holes which accept an externally threaded guide sleeve which also has internal threads to accept an externally threaded K-wire or olive wire which threads through the guide sleeve and into the tuberosity and/or humeral head to engage the humerus. These wires are threaded and will be threaded to the threaded guide and adjacent bone to hold the tuberosity and humeral head to the plate without any motion.

A second aspect of the system relates to an expandable cage member having a shaft which comprises a long central rod and a cage portion designed for intramedullary use which includes radially extending members such as ribs, flanges or arms that open outwardly to expand outwardly the volume circumscribed by the cage portion. The cage member can be used by itself in the humeral head (or at other places within the body), or to support, fix, reduce fractures, or buttress an associated orthopedic plate that is positioned against the lateral side of the humeral shaft. In the latter case, it extends through a hole in the plate which is configured to accept a cannulated screw driver that fits over the rod and pushes the runner at the intersection of the cage ribs. The screw driver is used to drive the runner downward on the central rod to expand the ribs of the cage outwardly. The runners slides over the rod and expands. Thus, the cage member extends into the bone such that the expandable cage portion extends in the direction of the humeral head under the subcortical cancellus bone so as to provide a buttress for the humeral head in the even if it is fractured.

The cage portion has a ferrule at one end and opposite of that, a runner which is journaled on an internal rod. A series of 4-6 extensive members, such as flexible longitudinal ribs extend between the ferrule and the runner. The ribs have a central angle which are open in the direction of the interior of the cage volume. As the runner is compressed toward the ferrule, the longitudinal distance on the rod between the ferrule and the runner is shortened which compresses the ribs along their length and sends the angled portion outwardly to increase the width of the figure circumscribed by the open ribs, in the manner that an umbrella is opened. The proper length of screw is measured with the special depth gage and proper size of locking screw is applied. The rod is cut flush to the plate when the cage member is passed through the plate to the desired distance and opening. Compression is achieved at the interface between the expandable cage member and the humeral head since the shaft of the expandable cage is held fixed to the plate with locking screw that also prevents it from backing out. In various embodiments, the cage can include alternative outwardly extending members that act to increase the volume of fixation in the head of the cage when the cage is in position within the bone and the cage is deployed.

### BRIEF DESCRIPTION OF THE DRAWINGS

FIG. 1a is a side end view of a proximal humeral plate and reduction and guide system;

FIG. 1b is a detail of a guide sleeve shown in FIG. 1a;

FIG. 1c is a side view of the drill guide body with the sleeve holes shown in phantom;

FIG. 2 is a side view of the proximal humeral plate of FIG. 1a with two distal screws in position to hold the plate against the lateral side of the humeral shaft and with an expandable cage member and associated screw driver in position to drive the cage member into position in the humeral head;

FIG. 3 is a side perspective view of the internal rod and expandable cage of the cage member;

FIG. 4 is a side view of the proximal humeral plate of FIG. 1a with five distal screws in position to hold the plate against the lateral side of the humeral shaft with five proximal screws and a deployed cage member to secure the proximal construct and inhibit varus subsidence of the humeral head;

FIG. 5a is a front lateral view of the proximal humeral plate of FIG. 1a on the lateral side of a proximal humerus;

FIG. 5b is a smaller diameter insert which can be used within an insert for the cage holes in the intermediate portion of the proximal humeral plate of FIG. 1a;

FIG. 5c is a side perspective view of a larger diameter insert that can be used to provide for an locking bone screw;

FIG. 5d is a detail of the larger diameter insert of FIG. 5c;

FIG. 6 is a side view of a second embodiment of the expandable cage member of the present invention including the shaft and internal rod and having arms that form an open expandable cage;

FIG. 7 is an end view of a cage as illustrated in FIG. 7, but with an increased number of arms and where the cage is deployed to open the volume;

FIG. 8 is a side view of the cage of FIG. 6 with the arms fully closed for insertion of the cage member through the plate and within the bone;

FIG. 9 is a third embodiment of the cage on the internal rod, but with a cage having hinged ribs where the hinges are located closer to the ferrule end of the cage;

FIG. 10 shows a first step in a method of surgery in accordance with the present invention in which a K wire applied through a guide to the proper depth while checking under fluoroscopy and then the guide is removed while the K wire is left in place;

FIG. 11 shows a second step of the method in which a drill is used to drill the bone to proper depth while checking under fluoroscopy;

FIG. 12 shows a third step in which both the drill and the K wire are removed and a drill hole in the proximal humerus remains;

FIG. 13 show a fourth step in which the cage member is inserted through the plate hole, and a screw driver is slide over the rod of the cage member and the screw driver is advanced to push the runner and expand the cage;

FIG. 14 shows a fifth step in which a depth gage is used to measure the length of the locking screw;

FIG. 15 shows a sixth step in which the internal rod is cut flush with the plate with a rod cutter; and

FIG. 16 shows a seventh step in which a proper size of locking screw is applied.

### DETAILED DESCRIPTION OF THE DRAWING

The present invention provides a system for the treatment of proximal humeral fractures to be presented in a surgical tray as a kit, and which provide a synergy among the various components. In particular, one or more orthopedic plates are provided which abut the exterior surface of the proximal end of a humerus in use. The plate 100 is shown in position adjacent the lateral surface of a humeral shaft 12 and aligned at one end with the lower portion of a humeral head 10 below the superior margin of greater tuberosity. An interior (relative to the bone) surface 104 of the plate includes a gently curving topography to abut a generalized version of the humerus. Various sizes can be provided to account for individual variations in size and length. The plate includes an exterior surface 102 with a relatively constant thickness defining a perimetral edge 103. This design accounts for maintaining a thickness which has as little bulk as needed based on biomechanical study, but provides the optimal strength characteristics, but it should be understood that the plate can include areas of more or less thickness or width where additional strength or area for through holes for fixation are warranted. Thus, in one design, the plate 100 can include an area 101 which is wider at the proximal end, an intermediate area, 104, and a narrower distal portion 105.

These areas also include through holes of varying diameters to account for various means of fixation. Thus, the distal shaft portion 105 includes dual diameter combi holes 106 which accommodate either locking or non-locking holes 106 for corresponding locking or non-locking or variable locking screws 107. The superior most area of the distal portion 105 includes a slot 108, such as for visibility or to accommodate a compression screw.

The intermediate portion 104 includes through holes 110 that include internal threads 111. These holes can be used with an expandable cage member 170 which includes a threaded shaft 178 that can be threaded through the internal threads 111 of the intermediate portion to feed the expandable cage member 170 into the cancellous portion within the humeral head. The cage member 170 is inserted in the closed position, and then deployed to create a secure anchor within the humeral head. This is useful, especially where the bone is of poor quality, or for certain fractures, or in instances where the risk of a varus displacement of the humerus could occur.

The proximal most portion 101 includes a series of through holes 112 of a first size to accommodate screws, pins, or k-wires and more peripheral smaller holes 114 which allow for fixation of soft tissue, such as ligaments or tendons using tissue anchors, suture, surgical tacks or wire.

The system of the present invention also includes a novel guide 120 which has a guide body 122, an extension 124 which includes an internal surface 123 that overlaps and mates with a small area of the proximal exterior surface 102 of the plate 100. This extension 124 has screw holes 126 that accommodate screws 125 which hold the drill guide 120 to the plate during use. The guide 120 includes an interior surface 179 that is curved to accommodate the curve of the lateral tuberosity of the humeral head. The edge of the guide includes a hook 136 which engages the exterior surface of the bone and secures the proximal end of the guide to the bone. Drill guide holes 130 having internal threads 132 at an outer portion 130 receive cylindrical drill guide sleeves 140 which include internal threads 144 in an internal bore 198. The lower exterior portion of the drill guide sleeves include external threads 142 which engage the internal threads 132 of the guide holes 130. Externally threaded k-wires 154 mate with the internal threads of the drill guide sleeves to feed these wires into the bone. These threaded wires engage the threads in the sleeve and the tuberosity and humeral head bone 119 to hold the guide to the bone. This enables the surgeon to use the guide for reduction in an area that is often difficult to successfully reduce and keep reduced. Similar externally threaded guide sleeves 148 can be used in the threaded through holes in the distal portion of the plate, and can receive threaded or unthreaded k-wires 149 which help to hold reduction during the implantation of the plate system.

The expandable cage member 170 includes a shaft 180 joined to the expandable cage 175. The member includes an internal rod 184 which extends through both the cage 175 and the shaft 180. The cage 175 has a runner 188 that can ride up or down on the internal rod 184, and a ferrule 190, optionally with a sharp edge for cutting through bone, and extending in the direction of the internal rod 184 and connected to both the ferrule and the runner there are a plurality of extensive members, here ribs 186 which each include a hinge 192. When the runner moves toward the ferrule on the internal rod, the length decreases and the ribs expand the cage outwardly, much like an umbrella. The cage member also includes a handle 182 such that the shaft can be driven through the cage member holes 110 and into the humeral head. When the cage is located as appropriate, the cage is deployed (i.e. opened outward). The proper length of locking screw is applied over the rod of the cage to retain the cage member 170 within the hole 110. The shaft and internal rod are cut at the surface of the plate 100. Alternatively, the cage member can be used in other applications, including different anatomical locations, with plate or intramedullary rods or other implants or without them. The screw driver can also be used to close the cage, for example, for removal.

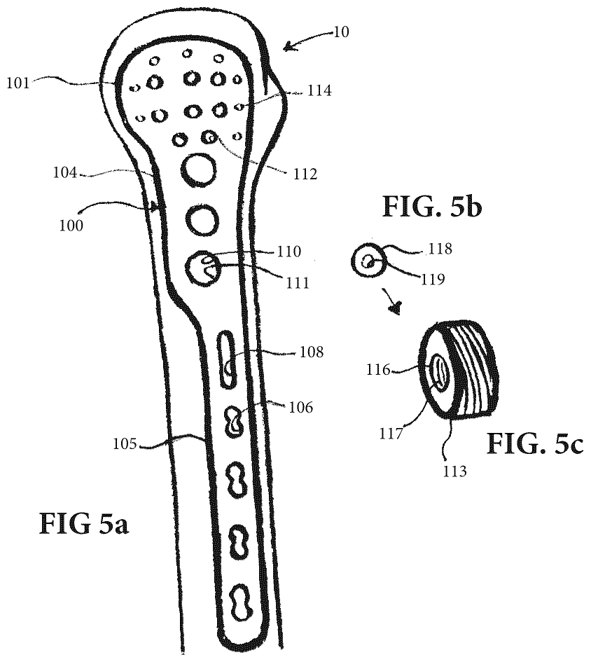

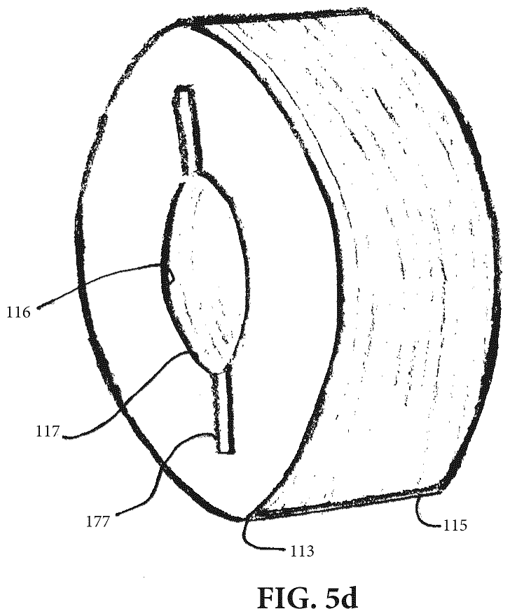

As an inventive additional feature, the larger cage member holes 110 can also be changed to accommodate a smaller diameter screw, such as a locking or non-locking screw customarily used in other standard screw holes in the associated plate. This is an advantage in allowing the freedom to select the means of fixation while reducing the inventory necessary to accomplish these choices. This is accomplished by inserting a threaded plug 113 in the hole 110. The plug also includes an internal hole 116 with threads 117 that can accommodate the external threads of a locking screw 107. In the detail shown in FIG. 5d the torque driving slot 177 in the hole and through the threads of the smaller insert is shown. The plate 100 has three larger cage member holes 110 in the intermediate portion 104 so that the surgeon has several options for the optimal placement of the cage member should they wish to use one with the plate. However, in the event that a surgeon wishes to use a screw in one or more of these holes the plug 112 can be inserted to allow a locking screw, and in the event they wish to use a pin, a second smaller externally threaded insert 118 having an internally threaded or unthreaded through hole 119 can be inserted in the threaded hole in the plug 112 to accommodate a smaller diameter fixator.

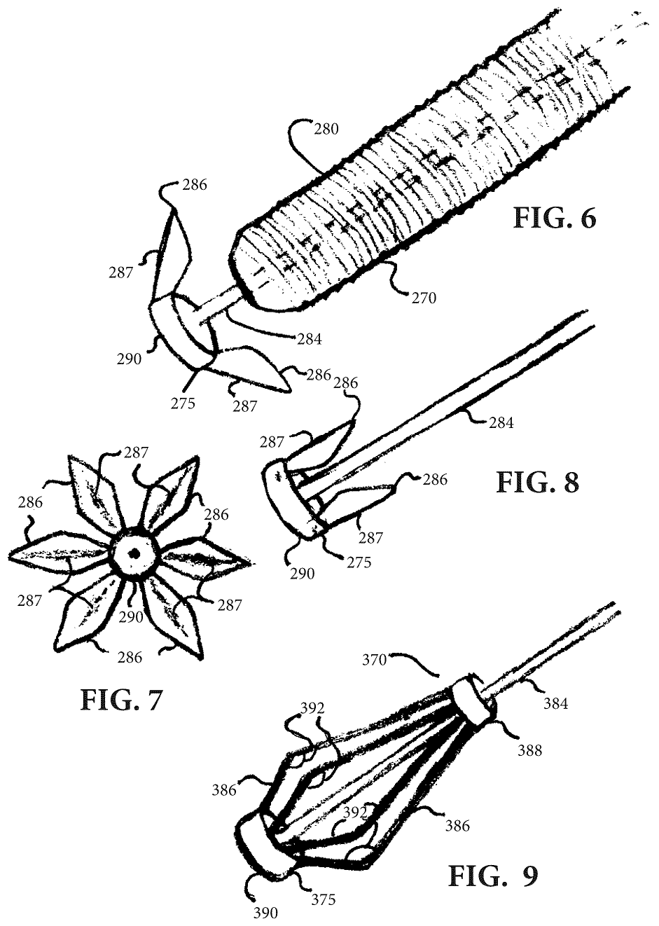

A second embodiment of the expandable cage member 270 of the present invention is shown in FIG. 6. Here, the expandable cage member 270 still includes a shaft 280 joined to the expandable cage 275. The member includes the internal rod 284 which extends through both the cage 275 and the shaft 280. In this instance, the cage 275 is an open cage having radially extending arms 286 instead of the rib members of the first embodiment. As before, the cage 275 includes the ferrule 290 and extending in the direction of the internal rod 284 and connected to the ferrule there are a plurality (i.e. from 2 to 8) of extensive members, now a plurality of arms 286. These arms are shown as having an outline as shown in FIG. 7 which is a diamond shape, and as is shown in FIG. 6 and FIG. 8, the diamond shape has a shape in the vertical dimension where the center axis 287 of the diamond represents a fold such that the arms angle away from the center axis where the diamonds now look like triangles in this view. The arms of the cage have a curve on the inner side which allows the screw driver to open the cage by a mechanism such as a convex flexible surface contained in the ferrule and activated to open the arms by the end of the screw driver. FIG. 9 shows a third embodiment of the cage member 370 which also includes a plurality of ribs 386 between a runner 388 and a ferrule 390 and which each include a hinge 392 to allow the enclosed volume to expand in relation to the change in the length between the runner 388 and the ferrule 390 along the internal rod 384 as for the first embodiment.

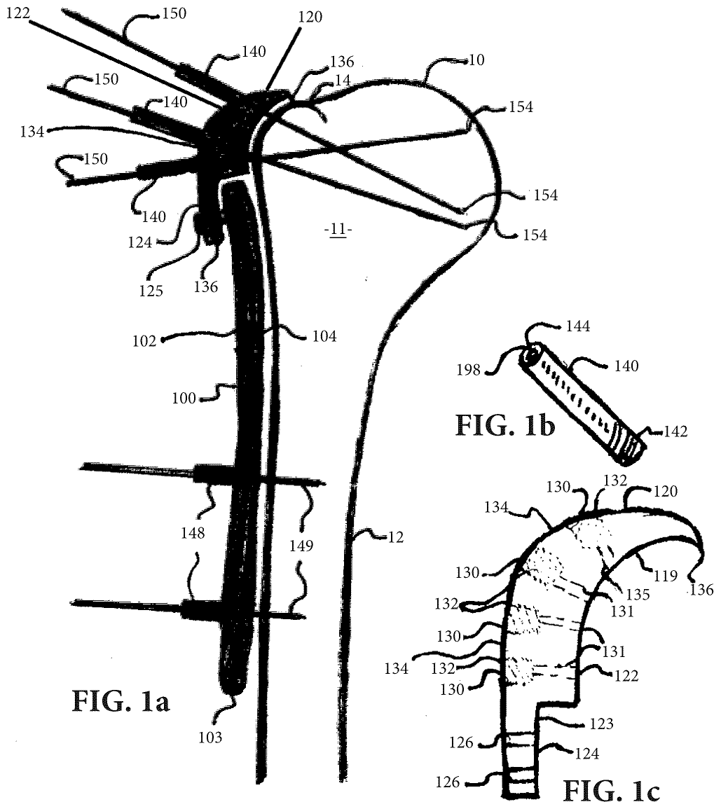

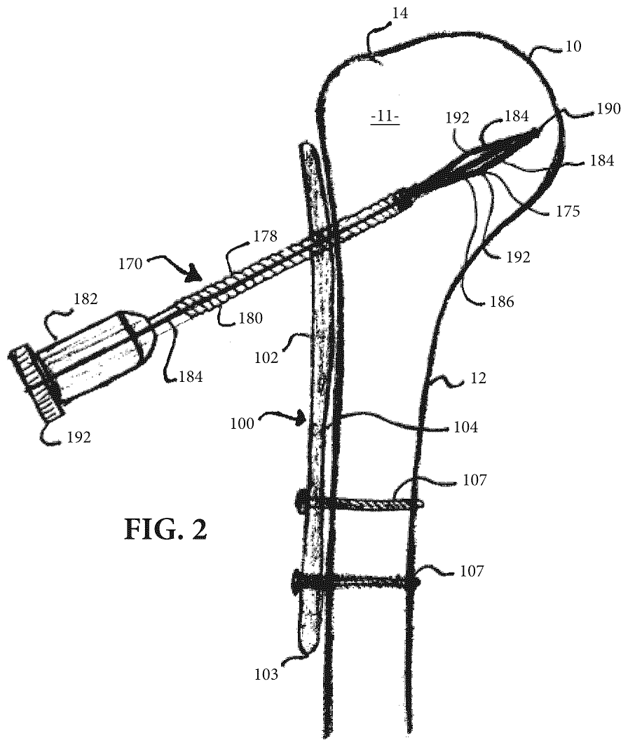

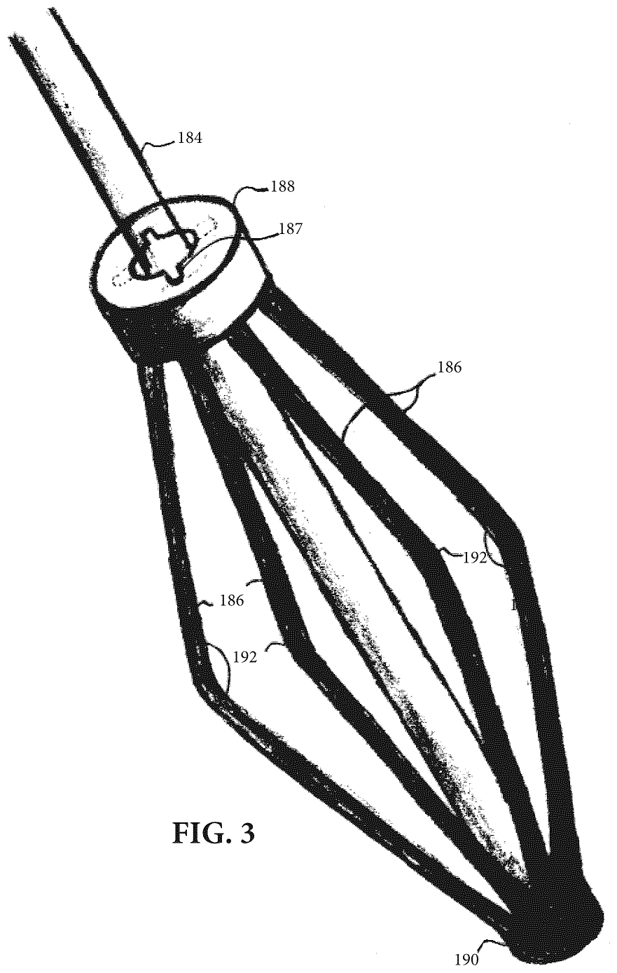

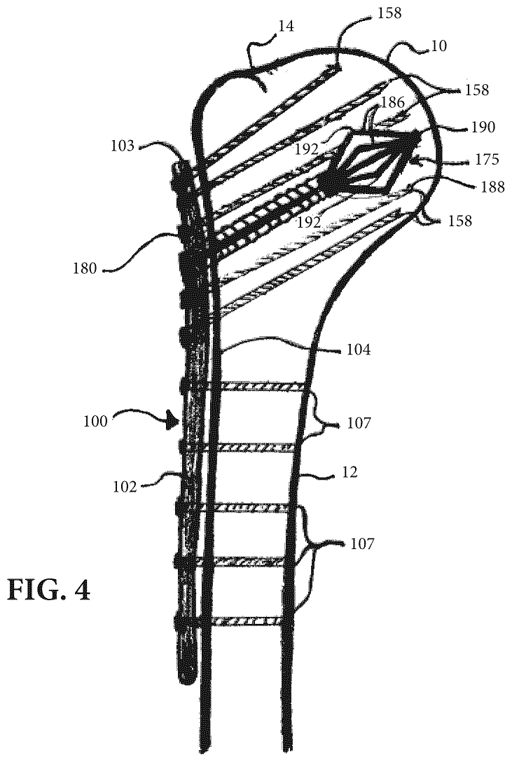

A procedure using the drill guide/reduction system of the present invention is illustrated with reference to FIGS. 1a, 2, and 4. FIG. 1a shows a portion of a humerus which has been prepped and exposed to allow for the placement of the orthopedic plate 100 along the lateral side of the humerus superior to the anatomic neck which joins the shaft and the head. The guide sleeves 148 are threaded into the distal screw holes in the distal portion of the plate, and either wires are driven directly through the sleeves, or pilot holes are made using the sleeves to guide the location and angle. K-wires are used to hold the distal portion of the plate to the humeral shaft.

The guide is placed on the exposed humeral tuberosity with the hook member tamped into the exterior surface of the humeral head at about the tuberosity of the humerus. The guide can be used to reduce the humerus and the guide is secured to the proximal end of the plate using a hole in the plate. Externally threaded guide sleeves are threaded into the guide holes, (which can be grasped to help reposition the guide with the extension overlapping the proximal end of the plate). These sleeves are used to drill pilot holes or to directly guide wires into the humeral head. Holes are selected in the drill guide so as to best capture fragments in expected fracture patterns.

A screw 107 is fed through a screw hole in the distal portion of the plate, the k-wires are removed, the drill sleeves 148 are disengaged, and a second bone screw 107 is fed through the distal portion of the plate to secure that end of the plate.

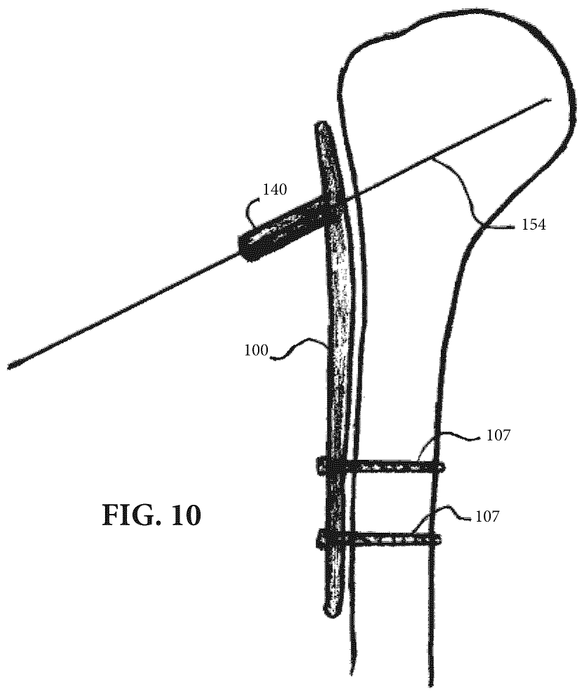

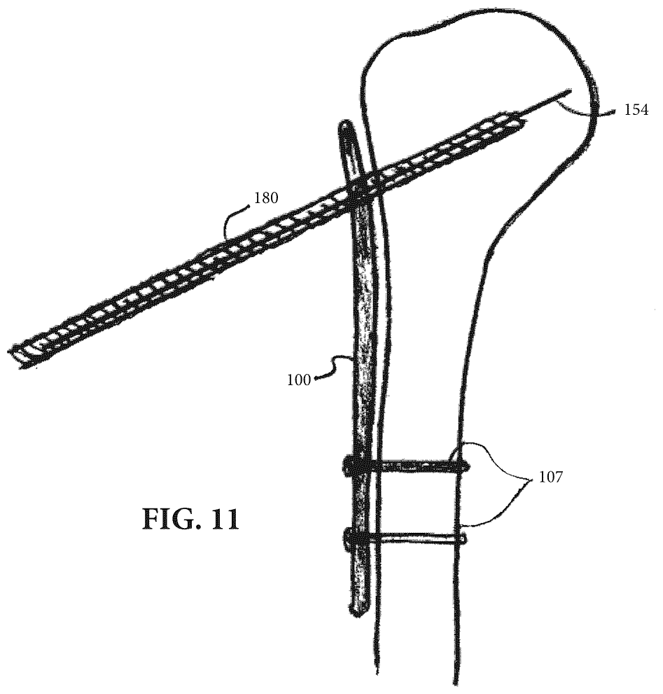

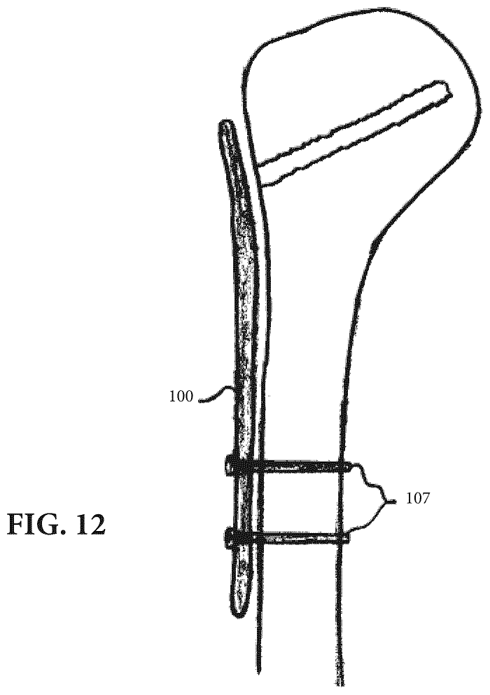

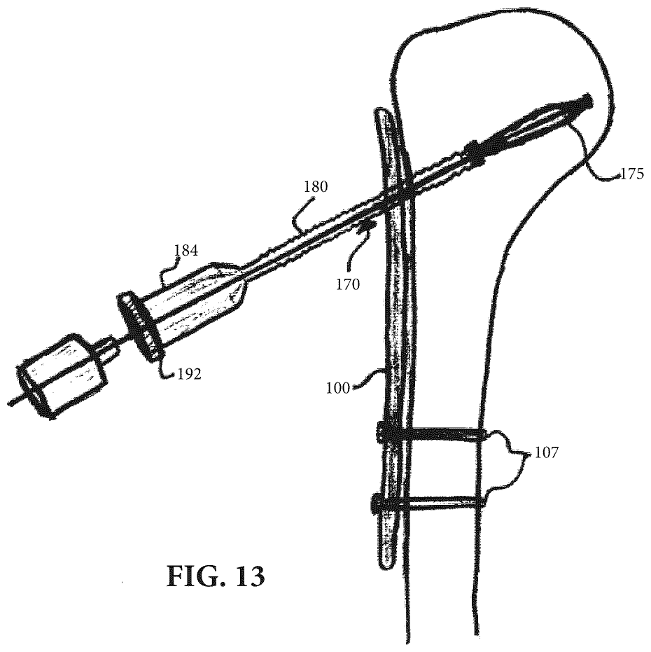

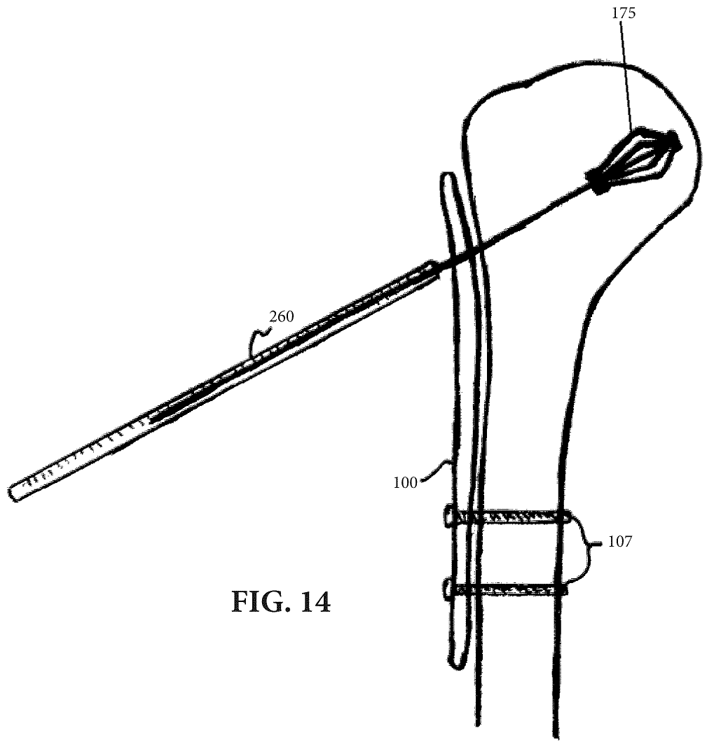

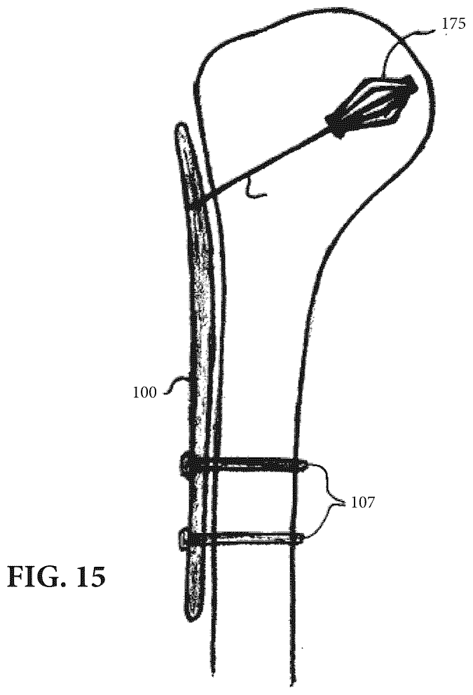

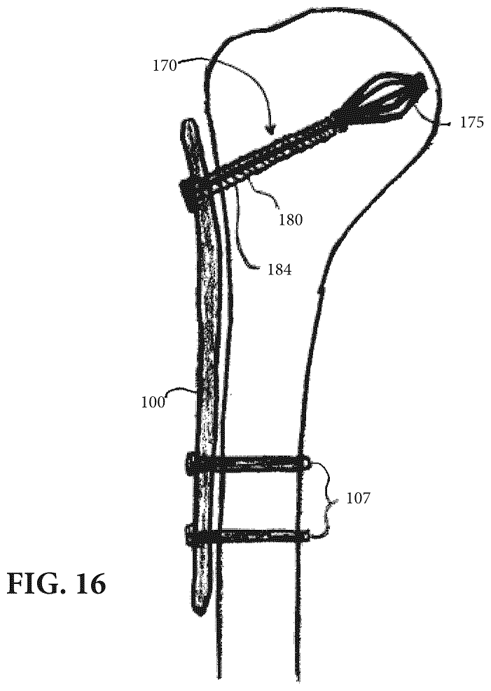

A procedure using the cage anchor system of the present invention is illustrated with reference to FIG. 10 to FIG. 16. In a first step of the procedure, following exposure of the site and placement of the plate 100 adjacent the bone in the anticipated placement and secured using distal screws 107, a guide tube 140 is placed in the appropriate hole in the plate 100 at a desired angle and then a K wire 154 applied through the guide tube 140 to the proper depth while checking under fluoroscopy for proper placement. Then the guide tube 140 is removed while the K wire 154 is left in place.

In a second step of the method, a drill is used to drill the bone to proper depth while checking under fluoroscopy for the appropriate placement. In a third step, both the drill and the K wire 154 are removed leaving a drill hole in the proximal humerus.

In a fourth step, (with or without the drill/reduction guide in place, depending on the judgement of the surgeon), the undeployed cage member 170 is inserted through one of the cage member holes 110 in the intermediate portion 104 of the plate 100 after having made the proper hole in humeral head with the designated drill. Based on imaging prior to or during the surgery, the depth of the cage member in the humeral head is estimated and/or checked, and when it is properly placed the screw driver portion 182 of the cage member assembly is used to shorter the length of the cage 175 and expand the ribs 186 by opening the hinges 192 using the turn member 192 which drives the runner over the rod. This is accomplished using the screw driver 182 which is slid over the rod 184 of the cage member 170 and the screw driver 182 is advanced to push the runner 188 and expand the cage 175. There is a screw driver that goes over the pin. There is another piece that get locked at the end of the pin to hold it still and prevent it from twisting while the surgeon is twisting the screw driver. In a fifth step in which a depth gage 260 is used to measure the length of the locking screw. The screw length is measured and in a sixth step in which a proper size of locking screw is applied. Subsequently, in a seventh step in which the internal rod is cut flush with the plate 100 with a rod cutter.

The other fixators, including proximal screws, intermediate screws, pins, or wires are placed, and soft tissue is also secured as required and the wound is closed.

What is claimed is:

1. A drill guide/reduction system for use with an orthopedic plate to fix a bone and having with a top surface and a bottom surface configured to face the bone, comprising the drill guide/reduction system comprising a body having a first surface contoured so as to have a portion that overlaps the plate and which can be secured to the plate and a portion having a second surface contiguous with the first surface having a concave curve which can envelop a portion of the bone, the second portion having a hook extending and in the direction of the concave curve and including at least one drill guide body through hole.

2. A drill guide/reduction system as set forth in claim 1, further including a drill guide tube which has a through hole and which is configured to mate with the drill guide body through hole.

3. A drill guide/reduction system as set forth in claim 2, wherein the through hole of the drill guide tube is threaded and the system further including a threaded k-wire having threads that are configured to mate with the threads of the k-wire.

4. A drill guide/reduction system as set forth in claim 2, wherein the exterior surface of the drill guide tube is threaded, the interior of the drill guide body hole is threaded and the exterior drill guide tube threads and the interior drill guide body threads are configured to mate.

5. A drill guide/reduction system as set forth in claim 4, wherein the exterior surface of the drill guide tube is threaded, the interior of the drill guide body hole is threaded and the exterior drill guide tube threads and the interior drill guide body threads are configured to mate.

6. A drill guide/reduction system as set forth in claim 1, wherein the drill guide body has a plurality of through holes and a plurality of guide tubes and wherein one or more of the guide tubes can be positioned at a variable angle within a through hole of the drill guide body.

7. A cage member for use within a bone comprising a shaft having an internal rod and a cage on the internal rod, the cage having a plurality of radially extensible members and a mechanism to extend the radially extensible members.

8. A cage member as set forth in claim 7, wherein the cage members has from 2 to 8 radially extensible members.

9. A cage member as set forth in claim 8, wherein the radially extensible members are radially symmetrical.

10. A cage member as set forth in claim 9, wherein the radially extensible members are one or more of ribs, arms or flanges.

11. A cage member as set forth in claim 10, further including a runner which is central to and connected to the radially extensible members and wherein the runner can longitudinally engaged to cause the radially extensible members to deploy outwardly.

12. A cage member as set forth in claim 11, wherein the radially extensible members are ribs having a hinge and are operatively connected at a first end a fixed length to the internal rod through a ferrule, and are operatively connected at a second end to a runner which can be positioned at a variable length from the ferrule on the internal rod and wherein the hinge is opened or closed by changing the position of the runner relative to the ferrule on the internal rod.

13. A plate system comprising a plate having an internally threaded hole of a first diameter and an externally threaded insert that engages the internal threads of the plate hole and the externally threaded insert includes a hole with a diameter smaller than the first diameter and that accepts a different diameter fixator.

14. A plate system wherein the externally threaded insert further includes internal threads and the system including a second insert having external threads which are configured to mate with the internal threads of the externally threaded insert.

15. A system for the fixation of proximal humeral fractures comprising a plate having an internal surface configured to be positioned adjacent a lateral surface of a humeral shaft and aligned at one end with the lower portion of a humeral head below the superior margin of greater tuberosity and having a bone facing surface including topography to about a generalized version of the humerus and including an exterior surface with a relatively constant thickness from the bone facing surface defining a perimetral edge and having a plurality of through holes configured to accept a fixator which is one or more of a locking screw, a non-locking screw, a variable angle screw, a threaded k-wire, and an expandable cage member.

16. A system for the fixation of a proximal humerus as set forth in claim 15, wherein the plate includes a proximal portion including a plurality of through holes of a first diameter having internal threads and further comprising at least one insert having external threads configured to mate with the internal threads of the plate and having an internal insert hole.

17. A system for the fixation of a proximal humerus as set forth in claim 16, wherein the internal insert hole is threaded and including a locking screw having external threads configured to mate with the internal insert hole threads.

18. A system for the fixation of a proximal humerus as set forth in claim 17, wherein the plate further includes a locking screw hole having internal threads configured to mate with the locking screw.

19. A system for the fixation of a proximal humerus as set forth in claim 15, wherein the expandable cage member has a cage with radially extensible members.

20. A system for the fixation of a proximal humerus as set forth in claim 19, wherein the radially extensible members are ribs.

21. A system for the fixation of a proximal humerus as set forth in claim 20, wherein comprising from 2 to 8 ribs.

22. A system for the fixation of a proximal humerus as set forth in claim 21, wherein the cage member includes an internal rod and the ribs each include a hinge and the ribs are joined to the internal rod at one end through a ferrule and are joined at the other end by a runner which is spaced a length away from the ferrule along the long axis of the internal rod, and the length between the runner and the ferrule can be changed which changes the volume within the cage formed by the ribs.

23. A system for the fixation of a proximal humerus as set forth in claim 15, further including a drill guide member having an internal curve which is configured to accommodate the proximal head of a humerus and the drill guide member includes a hook in the direction of the hook.

24. A method of surgery for fixation of a proximal humeral fracture comprising the step of exposing a lateral side of a proximal humerus, positioning a plate on the humerus, and using a drill guide having an internal curve with a hook to capture and reduce one or more fragments.

25. A method of surgery for fixation of proximal humeral fracture as set forth in claim 24, further including the step of using an intramedullary radially expandable cage within the humeral head.
